(19) United States
(12) Patent Application Publication
(10) Pub. No.: US 2026/0325633 A1
(43) Pub. Date: Oct. 22, 2026

(54) treating leukemia.

(71) Applicant: MENNI MENASHE ZINGER, Ashkelon (IL)

(72) Inventor: MENNI MENASHE ZINGER, Ashkelon (IL)

(21) Appl. No.: 19/185,287

(22) Filed: Apr. 22, 2025

(51) Int. Cl. A61K 40/11; A61K 9/00; A61K 31/4152; A61K 35/12; A61K 35/16; A61K 35/17; A61K 38/16; A61K 41/00; C12N 5/0783; C12N 13/00

(52) U.S. Cl. CPC A61K 40/11; A61K 9/0019; A61K 31/4152; A61K 35/16; A61K 35/17; A61K 38/16; A61K 41/00; C12N 5/0636; C12N 13/00; A61K 2035/124; A61K 2239/48

(57) ABSTRACT

At this invention I presents medication to leukemia and cancer/tumors.

Using quantum mechanics and inspiration zones and model and sample of t cells and duplicating the t cells as much as needed and after words injecting the duplicated/multiplied t cells/T cells into patient vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies. Multiplying can be to several plates/cans with using several luminums/rays/beams with using prism.

I assume that if the medication dipyrone 500 milligrams or more might lower the white cells at body/blood so it has potential to lower white cells in case of eruption at time of leukemia.

Using quantum mechanics and inspiration zones and model and sample of blood donation from different kinds/types and duplicating the blood donation after checking if it is sterile and match for specific patient/right type of blood as much as needed and after words injecting the duplicated/multiplied blood donated that was duplicated/multiplied into patient vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies and multiplying can be to several plates/cans with using several luminums/rays/beams with using prism.

Using quantum mechanics and inspiration zones and model and sample of rare proteins saving life materials/medications/antidots and duplicating the rare proteins saving life materials/medications/antidots as much as needed and after words injecting the duplicated/multiplied into patient with tumor/s/cancer vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies and multiplying can be to several plates/cans with using several luminums/rays/beams with using prism.

1. Using quantum mechanics and inspiration zones and model and sample of t cells and duplicating the t cells as much as needed and after words injecting the duplicated/multiplied t cells/T cells into patient with tumor/s/cancer vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies and multiplying can be to several plates/cans with using several luminums/rays/beams with using prism.

2. Using dipyrone medication 500-4000 milligrams a day for one week/7 days in order to treat leukemia.

3. Using quantum mechanics and inspiration zones and model and sample of blood donation from different kinds/types and duplicating the blood donation after checking if it is sterile and match for specific patient/right type of blood as much as needed and after words injecting the duplicated/multiplied blood donated that was duplicated/multiplied into patient vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies and multiplying can be to several plates/cans with using several luminums/rays/beams with using prism.

4. Using quantum mechanics and inspiration zones and model and sample of rare proteins saving life materials/medications/antidots and duplicating the rare proteins saving life materials/medications/antidots as much as needed and after words injecting the duplicated/multiplied into patient with tumor/s/cancer vain and the duplication/multiplying would be done by using magnetic/electro magnetic/electric field/t.v. waves frequencies/radio waves frequencies and transmitter and receiver when the receiver aimed towards the sample and the transmitter aimed towards empty sterile plate/can and creating the inspiration by those physical powers as magnetic/electro magnetic/t.v. waves frequencies/radio waves frequencies and multiplying can be to several plates/cans with using several luminums/rays/beams with using prism.
